(19) United States
(12) Patent Application Publication
(10) Pub. No.: US 2026/0334871 A1
(43) Pub. Date: Oct. 29, 2026

(54) Sleep Apnea and Hypoxia Detection and Mitigation System

(71) Applicants: Wisam Breegi, Woburn, MA (US); Mehmet Hadi Suzer, Kayapinar (TR)

(72) Inventors: Wisam Breegi, Woburn, MA (US); Mehmet Hadi Suzer, Kayapinar (TR)

(21) Appl. No.: 19/649,680

(22) Filed: Apr. 16, 2026

Related U.S. Application Data
(60) Provisional application No. 63/795,814, filed on Apr. 28, 2025.

(51) Int. Cl. A61B 5/00; A61B 5/01; A61B 5/08; A61B 5/11; A61B 5/113; A61B 5/1455; A61B 7/04; A61G 11/00; A61L 2/10; A61L 103/10; A61M 16/00; A61M 16/08; A61M 16/10

(52) U.S. Cl. CPC A61B 5/4836; A61B 5/01; A61B 5/08; A61B 5/1103; A61B 5/1126; A61B 5/113; A61B 5/14551; A61B 5/4818; A61B 5/7264; A61B 5/742; A61B 7/04; A61G 11/00; A61L 2/10; A61M 16/0003; A61M 16/0051; A61M 16/024; A61M 16/0875; A61M 16/109; A61B 2503/045; A61B 2560/0242; A61L 2103/10; A61M 2016/0027; A61M 2202/0007; A61M 2202/0208; A61M 2205/05; A61M 2205/18; A61M 2205/3303; A61M 2205/3306; A61M 2205/3327; A61M 2205/3331; A61M 2205/3368; A61M 2205/3375; A61M 2205/502; A61M 2205/52; A61M 2205/7545; A61M 2230/005; A61M 2230/20; A61M 2230/40; A61M 2230/50; A61M 2230/63; A61M 2240/00

(57) ABSTRACT

A sleep apnea and hypoxia detection and mitigation system is disclosed. The sleep apnea and hypoxia detection and mitigation system includes a contained enclosure, a plurality of sensors configured to monitor physiological and behavioral characteristics of the patient, an oxygen-air delivery module configured to automatically deliver an oxygen-air mixture to the patient, a physical stimulation module configured to stimulate the patient, and a processor configured to communicate with the plurality of sensors, and to control the oxygen-air delivery module and the physical stimulation module. The plurality of sensors includes at least one of the following sensors: the plurality of sensors comprises at least one of the following sensors: an optical sensor configured to capture optical data associated with the patient, a barometric pressure sensor, a temperature sensor, an audio sensor, an accelerometer, or a gyroscope. Additional features may include a caregiver notification module and a data storage module.

### CROSS-REFERENCE TO RELATED APPLICATION(S)

This application claims the benefit of priority to U.S. Provisional Patent Application No. 63/795,814, filed Apr. 28, 2025, the entirety of which is incorporated by reference herein.

### TECHNICAL FIELD

Various embodiments of the present disclosure relate generally to systems and methods for monitoring, detecting, and mitigating respiratory irregularities. More particularly, the present invention related to methods and systems for monitoring, detecting, and mitigating sleep apnea and hypoxia in patients.

### BACKGROUND

Respiratory irregularities such as sleep apnea and hypoxia are particularly prevalent and dangerous. Premature infant patients are especially vulnerable to brief interruptions in respiration or reductions in blood oxygen saturation, which can lead to developmental impairment, neurological injury, or organ damage if not promptly addressed. Moreover, it is important to provide a stable and clean environment for such patients.

Conventional respiratory irregularities monitoring systems are primarily passive. Existing solutions typically measure one or more physiological parameters—such as blood oxygen saturation or heart rate—and generate alarms when a parameter crosses a predefined threshold. A challenge for such conventional systems is potential high false positive alarms when the systems rely on simple monitoring parameters and thresholds. Additionally, these systems rely on caregiver intervention after respiratory irregularities have already begun, rather than actively preventing or mitigating respiratory episodes at an early stage.

The present disclosure is directed to overcoming one or more of these above-referenced challenges.

### SUMMARY OF THE DISCLOSURE

In some aspects, the embodiments described herein related to a system for monitoring, detecting, and mitigating respiratory irregularities, including a contained enclosure, a plurality of sensors configured to monitor physiological and behavioral characteristics of the patient, where the plurality of sensors includes at least a blood oxygen detector, an oxygen-air delivery module configured to automatically deliver an oxygen-air mixture to the patient, where the oxygen-air delivery module supplies an oxygen-air mixture to the contained enclosure, the oxygen-air delivery module further includes: an oxygen-air mixture recycling unit configured to recirculate air from within the contained enclosure, a conduit configured to connect the oxygen-air mixture recycling unit to the contained enclosure, a sterilization unit located within the conduit and configured to sterilize the oxygen-air mixture, a fan configured to maintain and adjust the air flow into the enclosure, a physical stimulation module configured to stimulate the patient, and a processor configured to communicate with the plurality of sensors, and to control the oxygen-air delivery module and the physical stimulation module.

In some aspects, the plurality of sensors includes at least one of the following sensors: an optical sensor configured to capture optical data associated with the patient, a barometric pressure sensor, a temperature sensor, an audio sensor, an accelerometer, or a gyroscope.

In some aspects, the system further includes a caregiver notification module configured to generate an alert upon a status change of the oxygen-air delivery module or the physical stimulation module.

In some aspects, the oxygen-air delivery module further includes a nebulizer.

In some aspects, the oxygen-air delivery module further includes an oxygen actuator configured to adjust a concentration of oxygen in the oxygen-air mixture.

In some aspects, the physical stimulation module includes at least one of a vibration device, an airflow emitter, an auditory stimulus device, or a visual stimulus device.

In some aspects, the system further includes a data storage module configured to record sensor data and system operation actions.

In some aspects, the sterilization unit includes an ultraviolet light source.

In some aspects, the sterilization unit further includes at least one air filter positioned downstream or upstream of the ultraviolet light source.

In some aspects, the air filter includes high-efficiency particulate air (HEPA) filter.

In some aspects, the processor is configured to control the oxygen-air delivery module based on a result from a machine learning model, wherein the machine learning model comprises deep neural networks, kernel-based methods, and ensemble techniques.

In some aspects, the caregiver notification module is configured to transmit the alert to at least one of a display, a mobile device, or a remote monitoring station.

In some aspects, the embodiments described herein related to a method for detecting, monitoring, and mitigating respiratory irregularities in a patient, the method includes: continuously monitoring physiological and behavioral characteristics of the patient using a plurality of sensors, analyzing, by a processor, measurements from the plurality of sensors, determining, based on the analyzing, whether an impending or early-stage respiratory irregularity is present, and automatically initiating a first mitigation response by delivering an increased oxygen-air mixture to the patient via an oxygen-air delivery module when the impending or early-stage respiratory irregularity is detected.

In some aspects, the physiological and behavioral characteristics include at least one of blood oxygen level, respiratory activity, chest movement, abdominal movement, body orientation, body temperature, acoustic signals, eye movement, or patient restlessness.

In some aspects, determining an impending or early-stage respiratory irregularity includes: determining whether a blood oxygen level drops below a predefined threshold, determining whether one or more additional physiological or behavioral characteristics of the patient are abnormal when the blood oxygen level drops below the predefined threshold, and determining, based on the analyzing, that the impending or early-stage respiratory irregularity is present.

In some aspects, determining whether an impending or early-stage respiratory irregularity is present includes evaluating an output of a machine learning model, where the physiological and behavioral characteristics are provided as inputs to the machine learning model.

In some aspects, the method for detecting, monitoring, and mitigating respiratory irregularities in a patient further includes initiating a second mitigation response when the first mitigation response fails to resolve the respiratory irregularity within a predetermined time period, where the second mitigation response includes applying at least one of vibration, airflow, light, or sound stimuli.

In some aspects, the method for detecting, monitoring, and mitigating respiratory irregularities in a patient further includes sterilizing the oxygen-air mixture prior to delivering the oxygen-air mixture to the patient.

In some aspects, sterilizing the oxygen-air mixture includes exposing the oxygen-air mixture to ultraviolet radiation.

In some aspects, sterilizing the oxygen-air mixture comprises filtering the oxygen-air mixture by a high-efficiency particulate air (HEPA) filter.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts a block diagram showing an overview of a system for monitoring, detecting, and mitigating respiratory irregularities in a patient, according to one or more embodiments.

FIG. 2 depicts a schematic view of a system for monitoring, detecting, and mitigating respiratory irregularities in a patient, according to one or more embodiments.

FIG. 3 depicts a schematic view of an open-air delivery module of a system for monitoring, detecting, and mitigating respiratory irregularities in a patent, according to one or more embodiments.

FIG. 4 depicts a schematic view of sensors for monitoring, detecting, and mitigating respiratory irregularities in a patient, according to one or more embodiments.

FIG. 5 depicts a flowchart outlining the steps of a method for monitoring, detecting, and mitigating respiratory irregularities in a patient, according to one or more embodiments.

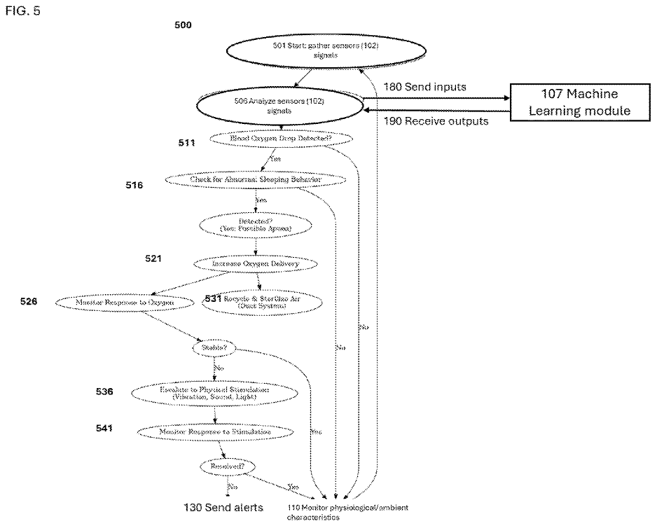

FIG. 6 depicts a flowchart outlining machine learning processes used for monitoring and detecting respiratory irregularities in a patent, according to one or more embodiments.

### DETAILED DESCRIPTION OF EMBODIMENTS

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value. In this disclosure, unless stated otherwise, any numeric value may include a possible variation of ±10% in the stated value.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

Various embodiments of the present disclosure relate generally to systems and methods for monitoring, detecting, and mitigating respiratory irregularities. More particularly, the present invention related to methods and systems for monitoring, detecting, and mitigating sleep apnea and hypoxia in patients.

Respiratory irregularities such as sleep apnea and hypoxia are particularly prevalent and dangerous. Premature infant patients are especially vulnerable to brief interruptions in respiration or reductions in blood oxygen saturation, which can lead to developmental impairment, neurological injury, or organ damage if not promptly addressed. Moreover, it is important to provide a stable and clean environment for such patients. When respiratory irregularities have already begun, it would be beneficial to actively prevent or mitigate respiratory episodes at an early stage.

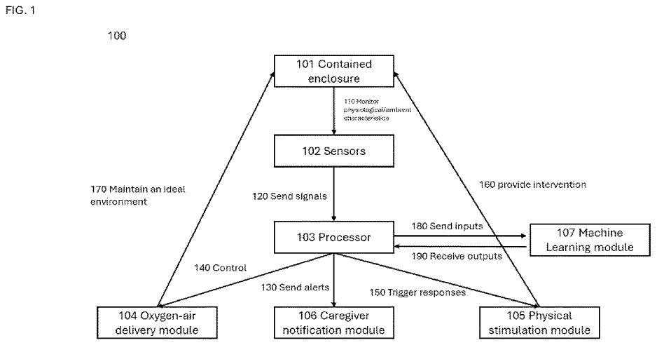

The present disclosure is directed to a system and method for monitoring, detecting, and mitigating respiratory irregularities in a patient (471). FIG. 1 shows an overall system (100) and its components in one embodiment. The overall system (100) may include a contained enclosure (101). The overall system may further include a plurality of sensors (102) which may include, for example, a blood oxygen detector (421), a processor (103), an oxygen-air delivery module (104), a physical stimulation module (105), a caregiver notification module (106), and a machine learning module (107). In one embodiment of the invention, the oxygen-air delivery module may maintain an approximately ideal environment (170) of the contained enclosure (101) such as by supplying, adjusting, and circulating oxygen or an oxygen-air mixture, such as, for example, sterilized oxygen. In one embodiment of the invention, sensors (102) may continually monitor the physiological/ambient characteristics of the patient (471) in the contained enclosure (101) as shown in step (110). Sensors (102) may send signals (120) to the processor (103) as shown in step (120). In one embodiment of the invention, upon receiving data from the sensors (102), the processor (103) may determine if respiratory irregularities have occurred or are likely to occur. In response to potential respiratory irregularities, processor (103) may, for example, send alerts to caregiver notification module (106) as shown in the step (130), trigger responses of physical stimulation module (105) to provide intervention (160) to the contained enclosure (101) as shown in step (150), and/or control (140) oxygen-air delivery module (104) to adjust the oxygen flow to the contained enclosure (101) as shown in step (140). In one embodiment of the invention, processor (103) may send inputs (180) to the machine learning module as shown in step (180) and receive outputs from the machine learning module (107) as shown in step (190). In one embodiment of the invention, machine learning module (107) may be, for example, another standalone processor, cloud-based server, or logic units in the processor (103).

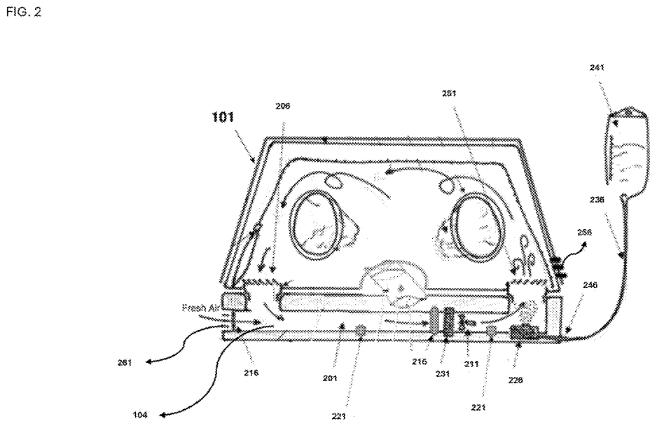

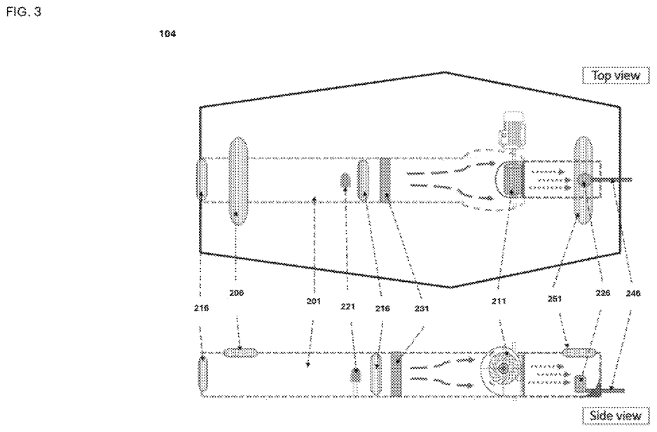

FIG. 2 and FIG. 3 show a detailed view of oxygen-air delivery module (104) and its connection with the contained enclosure (101). In one embodiment of the invention, the oxygen-air delivery module (104) may include a single-duct air pathway (201). This single-duct air pathway may serve as a main route for oxygen-air intake, sterilization, humidification, and recirculation. Air or oxygen-air may be drawn from the outside, via fresh air duct (261), or from within the contained enclosure (101) through a ventilation outlet (206) and pulled into the single-duct air pathway (201) by an upstream fan (211). While within the single-duct air pathway (201) the oxygen-air may then flow through a series of additional components, such as, for example, a filter (216) and a UV light (221), which may be positioned within the single-duct air pathway (201), so as to, for example, neutralize and trap airborne pathogens. In one embodiment of the invention, the filter (216) may be a High-Efficiency Particulate Air (HEPA) filter. In one embodiment of the invention, the filter (216) may be located closer to ventilation inlet (251) than the UV light (221). In one embodiment of the invention, the filter (216) may be located further from ventilation inlet (251) than the UV light (211). In one embodiment of the invention, the UV light (211) may be located in between the first filter (216) and the second filter (216). In a further embodiment of the invention, single-duct air pathway (201) may include a humidification segment, which may include, for example, a nebulizer (226) to convert a fluid, such as, for example sterile saline or another sterile fluid, into a mist, a heating element (231) to vaporize the mist, and a fluid conduit (236) such as may be connected to an external fluid reservoir (241) via an inlet connection (246). The now-treated air may then be routed back into the contained enclosure (101) through a ventilation inlet (251), which may be positioned near the head of the patient (471), thus completing a continuous and contained air loop.

In one embodiment of the invention, the patient (471), typically a neonatal infant, may be placed within this contained enclosure (101) so as to provide a controlled space for monitoring and atmospheric oxygen delivery. One example of such an environment includes, but is not limited to, a Patient (471) Intensive Care Incubator (NICI™), which is a disposable, expandable, portable, and lightweight microenvironment designed to maintain a medically sterile and controlled atmosphere. The NICI™ may serve as a patient (471) incubator suitable for both stationary and transport use. Further details regarding the NICI™ can be found in U.S. Pat. Nos. 10,251,801 and 11,439,554, which are incorporated herein by reference. Other embodiments of the invention may use an oxygen box or nasal cannula as alternatives to a contained environment.

In one embodiment of the invention, oxygen may be supplied through an adjustable flow regulator (oxygen actuator) (256), which may be controlled by the processor (103) to deliver determined concentrations of gasses, such as, for example, oxygen, air, or other gasses, based on real-time sensor data. In one embodiment of the invention, the oxygen actuator (256) may comprise an electronically or mechanically controlled device capable of modulating gas flow parameters, including flow rate, pressure, and oxygen/gas concentration. In one embodiment of the invention, the oxygen actuator (256) may include, for example, proportional valves, mass flow controllers, variable-speed compressors, or electronically regulated pressure systems. The oxygen may be supplied directly or indirectly into the contained enclosure (101) and then recirculated through an oxygen-air mix recycling process done by the air recycling and sterilization module (105). One embodiment of the invention may feature a single-duct air pathway (201) integrated into the contained enclosure (101) that operates as an air pathway (201) that may include devices to sterilize, humidify, and recirculate the air passing through it. The single-duct air pathway (201) may include ventilation and humidity inlets (251) and outlets (206), a fan (211) to maintain consistent air movement, a tankless humidification system (which may include, for example, a nebulizer (226) and a heating element (231)), and a combination of ultraviolet lighting (221) for air sterilization and a filter (216) for filtration. Air may be drawn from outside the contained enclosure (101) through fresh air duct (261) or from within the contained enclosure (101) through a ventilation outlet (206) and pulled into the single-duct air pathway (201) by an upstream fan (211) This fan, which may be located near the ventilation and humidity inlets (251), may provide constant pressure to ensure consistent air flow. Next, the air may pass through the tankless humidification system where the nebulizer (226) may convert sterile water, saline, or other liquid, from an external reservoir (241) into droplets. The heating element (231) may then heat the droplets into vapor by vaporizing the mist, such that the recycled air may be properly humidified to possibly protect the patient (471) from airway irritation. The humidification system may be connected to a fluid source, such as, for example, an external fluid reservoir (241), via a fluid conduit (236), allowing for continuous replenishment of liquid such as, for example, an uncontaminated/sterile water supply. Finally, the treated air may be redirected back into the enclosure through the ventilation inlet (251). This completes the circulation loop, allowing for continuous recycling and reintroduction of clean, humidified, oxygen-regulated air while protecting the patient (471) from external contaminants.

In one embodiment of the invention, sterilization of circulating air/gasses may be achieved through a combination of, for example, a UV light (221) and a filter (216). One embodiment of the invention may utilize UV lighting along the single-duct air pathway (201). Effective UV sterilization may require, for example, an extended duration of exposure to the UV light (221), beyond the time it takes for the air to pass through the single-duct air pathway (201) over the UV light (221). To reduce the time necessary for the sterilization, one embodiment of the invention may also introduce filters (216) positioned within the effective range of a UV light source (221) so as to provide sterilization of filters (216). In one embodiment, the filter (216) may be, for example, a HEPA filter or other appropriate type of filter. The recycled air may first pass through the UV light, and thereafter may flow through a downstream HEPA filter (216). In another embodiment of the invention, the recycled air may pass through additional HEPA filter (216) before it passes through the UV light (221). While the filter (216) captures airborne contaminants, the UV light (221) continuously irradiates the upstream face of the filter (216) that is facing the light, thus actively neutralizing pathogens and harmful particulates trapped on its surface. As a result, the combination of UV sterilization and high-efficiency filtration may result in the air passing through the system having reduced potentially harmful materials and airborne pathogens. Additionally, the UV light source (221) may maintain continuous sterility along the inner surfaces of the single-duct air pathway (201). This integrated process may ensure that all recirculated air is sterilized prior to being reintroduced into the environment. Multiple layers of filters (216) and UV light (221) may be employed to further enhance overall system effectiveness.

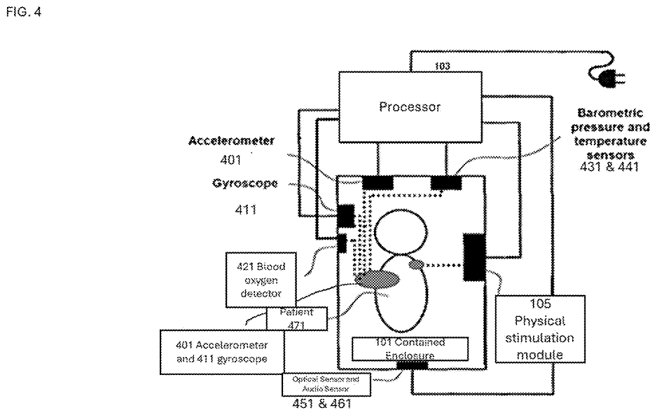

In one embodiment of the invention as shown in FIG. 4, with the patient (471) in the contained enclosure (101) or while connected to an alternative source of oxygen, the system may employ a plurality of sensors (102) to monitor physiological characteristics and/or environmental characteristics of contained enclosure (101). In one embodiment of the invention, such physiological/environmental characteristics may include, for example, orientation, movement, ambient pressure, ambient temperature, respiration rate, heart rate, and internal acoustic activity, in addition to blood oxygen levels, and other potential indicators of a respiratory irregularities such as an apnea or hypoxic episode. Sensors (102) may be affixed to the patient (471) such as, for example, via a flexible, adhesive wearable patch. Sensors (102) may alternatively be associated with the patient (471) in a contactless manner using motion sensitive sensors positioned to observe the patient (471)'s movements or activity. In some embodiments of the invention, sensors (102) may include, for example: accelerometer (401), gyroscope (411), blood oxygen detector (421); barometric pressure sensor (431), temperature sensor (441), audio sensor (451), or optical sensor (461). Typically, the more sensors (102) that the system (100) employs, the more possible indicators of respiratory irregularities may be detected, possibly leading to quicker and more accurate detection of respiratory irregularities.

In one embodiment of the invention, the blood oxygen detector (421) may include, for example, a pulse oximeter. In one embodiment of the invention, the audio sensor (451) may include, for example, a digital microphone or a stethoscope. In one embodiment of the invention, the temperature sensor (441) may include, for example, a digital thermometer with contact probes, a non-contact infrared thermometer, or a wearable continuous temperature monitor. In one embodiment of the invention, the optical sensor may include, for example, a camera (RGB, CMOS, CCD), an Infrared sensor, a thermal imaging sensor, a photodiode sensor, a light sensor, a time-of-flight sensor, or a depth sensing sensor.

In one embodiment, the system (100) may monitor the blood oxygen level of the patient (471) via blood oxygen detector (421) continuously.

In one embodiment of the invention, respiratory activity may be monitored through multiple sensors (102), including, for example, accelerometers (401) and gyroscopes (411) that measure chest and abdominal movements associated with breathing. In further embodiment of the invention, these sensors may be positioned directly on the patient (471)'s body or integrated into the surface of the contained enclosure.

In one embodiment of the invention, an audio sensor (461) may be used to detect breath and heart sounds to identify irregularities in respiratory patterns. In another embodiment of the invention, a temperature sensor (441) may monitor the patient (471)'s body temperature for signs of distress that could indicate respiratory irregularities such as apnea or other respiratory complications. In another embodiment of the invention, an optical tracking sensor (451) may monitor eye movement and restlessness.

In one embodiment of the invention, a temperature sensor (441) may monitor the ambient temperature of the contained enclosure (101). In another embodiment of the invention, a barometric pressure sensor (431) may monitor the ambient pressure of the contained enclosure (101).

Additional sensors (102) may be added to the system (100) to track various potential indicators of respiratory irregularities such as oncoming sleep apnea or hypoxic episodes.

Sensors (102) may send signals to the processor (103) for further analysis and control of components of the system (101), as shown in FIG. 1. After receiving signals from sensors (102), processor (103) may implement method (500) for monitoring, detecting, and mitigating respiratory irregularities. The method (500) may begin with the system (100) continuously or periodically gathering signals from sensors (102) in step (501). These signals may be transmitted to the processor (103) by each of the sensors (102) continuously, synchronously, asynchronously, or by any other timing according to method (500). Alternatively, processor (103) may request readings from each of the sensors (102) as needed or may poll sensors (102) according to any other timing as needed by method 500.

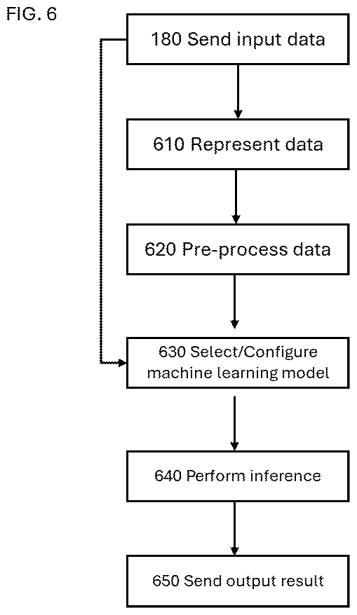

In one embodiment of the invention, after receiving signals from sensors (102), processor (103) analyze sensors signals in step (506) and prepare for following steps of method (500) to monitor, detect, and mitigate respiratory irregularities. In one embodiment of the invention, step (506) may include, for example, preprocessing the raw sensor signals including filtering, smoothing, segmentation, artifact reduction, and other common signal processing steps. In another embodiment of the invention, step (506) may include performing real-time analysis of blood oxygen levels. In another embodiment of the invention, step (506) may further include sending inputs to machine learning module (107) and receiving outputs from machine learning module (107). Details of machine learning module (107) and associated operations are discussed in detail below with respect to FIG. 6.

In one embodiment of the invention, the processor (103) may detect a drop in oxygen saturation below a predefined early-warning threshold in step (511). Such detection may be made when oxygen saturation falls below the predefined early-warning threshold for any length of time, or after a predetermined length of time so as to filter transient fluctuations of oxygen saturation. When a drop in oxygen saturation is detected, processor (103) may proceed with step (516), in which processor (103) may conduct a corroborative analysis using the remaining sensors (102) data such as to check for other abnormal sleeping behaviors. In step (516) processor (103) may, thus, verify whether the drop in oxygen is part of a broader respiratory irregularity indicative of an impending or active sleep apnea or hypoxia episode. For example, reduced chest movement, abnormal posture, stillness, or lack of typical behavioral responses may confirm that the oxygen drop is not an isolated fluctuation but rather part of a concerning pattern. The processor (103) may, thus, both evaluate correlations between blood oxygen saturation levels, and other physiological/ambient characteristics, such as movement patterns, and respiratory behaviors to determine whether an apnea event or hypoxia is imminent. By analyzing multiple indicators rather than relying solely on blood oxygen levels, the system (100) may identify concerning trends earlier in an episode, possibly allowing for a response before significant oxygen deprivation occurs. This holistic approach not only may enable earlier intervention but also may enhance accuracy in detecting true apnea events, reducing the likelihood of false alarms or missed episodes. In one embodiment, the processor (103) may use predefined threshold-based method to detect early signs of respiratory distress.

When abnormal sleeping behavior is detected in step (516), processor (103) may control oxygen-air delivery module (104) to mitigate risks. For example, in one embodiment of the system, if abnormal indicators are found in conjunction with the blood oxygen level fluctuation, the processor (103) may then initiate an initial mitigation response.

In one embodiment of the system, the initial mitigation response may include, in step (521), providing increased measured oxygen to the patient (471). This may be, for example, an automatic adjustment of oxygen delivery, increasing the concentration as needed while continuously monitoring the patient (471)'s physiological/ambient characteristics. In one embodiment of the invention, after increasing the oxygen supply, the processor (103) may recycle and sterilize air via oxygen-air delivery module (104). The processor (103) may then resume monitoring the blood oxygen level of the patient (471) and patient's physiological/ambient characteristics. Ideally, blood oxygen may then stabilize within a normal range, and the patient (471) may resume a normal breathing pattern. However, if the increased oxygen supply does not stabilize blood oxygen at a normal level or if abnormal breathing persists after a predetermined period of time, then the processor (103) may initiate a physical stimulation response in step (536). In one embodiment of the invention, the physical stimulation response (536) may be accomplished through physical stimulation module (105). For example, in one embodiment of the invention, a vibration module may be worn by the patient (471) to provide gentle tactile and/or respiratory stimulation to prompt proper breathing and cut off an impending episode. In addition to vibration, the physical stimulation module (105) may include auditory stimuli, such as soft tones or white noise, visual stimuli, such as flashing lights, or an air pulse emitter to gently stimulate the patient (471) with an oxygen/air mix across the patient (471)'s face.

In one embodiment of the invention, the system (100) may also include a caregiver notification module (106). Caregiver notification module (106) may generate an alert to notify caregivers of a potential episode and any response initiated by the system. The system may be configured to communicate alerts to an external monitoring station, mobile device, or hospital alert system by any suitable wired or wireless network, such as, for example, WiFi, low energy Bluetooth, radio, Internet, wide-area network (WAN), local-area network (LAN), etc. Alerts may alternatively or concurrently be transmitted via an integrated display screen integrated into the contained enclosure (101), via a loudspeaker integrated into the contained enclosure (101), via indicator lights integrated into the contained enclosure (101), via buzzers or other noisemakers integrated into the contained enclosure (101), or by any other suitable means. Wired or wireless network connectivity may further enable the system (100) to store data in a networked storage device, such as, for example, cloud storage, such that the data is available for use further analysis to improve the operation of the system (100), such as via machine learning processes discussed in detail below with respect to FIG. 6.

Upon verification of an irregularity, the system (100) may initiate a first-stage mitigation response, such as, by activating the oxygen-air delivery system (104). In response, oxygen-enriched, filtered, and humidified air may be delivered into the patient (471)'s environment within the contained enclosure (101). If this intervention successfully restores normal sensor readings, the system may return to the monitoring phase. However, if the abnormal patterns persist beyond a predetermined time window, the system may escalate to deploying physical stimulation. This may include activating vibration pads, localized airflow emitters, lights, or auditory stimuli intended to gently rouse the patient (471) and encourage resumed breathing.

In one embodiment of the invention, throughout the process, caregivers may be notified via caregiver notification module 106, and air may be sterilized and recycled via oxygen-air delivery module (104). In one embodiment of the invention, the caregiver notification module may further log the full sequence of events, including the oxygen drop, sensor data, system actions, and outcome, into cloud-based storage or local storage. This data may later be used for retrospective analysis or to improve detection algorithms of machine learning module (107), as discussed below with respect to FIG. 6.

FIG. 6 shows a method for improving the operation of system (100) to detect early signs of respiratory distress via machine learning models, such as may be employed by the machine learning module (107). In one embodiment of the invention, the steps to implement machine learning models may include sending input data to the models (180), representing data (610), pre-processing data (620), selecting/configuring a machine learning model (630), performing inferences (640), and sending output results (650).

In one embodiment of the invention, input data sent to the machine learning module (107) as shown in step (180) may include, for example, multimodal physiological and environmental signals such as respiratory waveforms, blood oxygen saturation (SpO₂), heart rate, airflow, motion/actigraphy, and acoustic signals, etc.

In one embodiment of the invention, after receiving the input data, the machine learning module (107) may, in step (610) represent the received data in any other form as needed for further processing, such as, for example, as continuous time-series, segmented windows, or derived feature vectors.

In one embodiment of the invention, pre-processing the received data in step (620) may include, for example, signal denoising, filtering, normalization, temporal segmentation, and transformation into time-frequency representations (e.g., FFT, spectrograms), as well as statistical or learned feature extraction. In another embodiment of the invention, end-to-end models may operate directly on raw signals included in the received data. In another embodiment of the invention, represented data in step (610) may ne fed directly into machine learning models in step (640).

In one embodiment of the invention, machine learning models in step (640) may include deep neural networks (e.g., CNNs, RNNs, LSTMs, transformers), kernel-based methods (e.g., SVMs), and ensemble techniques (e.g., random forests, gradient boosting). In one embodiment of the invention, machine learning module may be implemented hybrid architectures, such as by combining multiple machine learning models.

In one embodiment of the invention, training the machine learning model (107) may be performed using supervised, semi-supervised, self-supervised, or unsupervised approaches, with datasets derived from clinical recordings, controlled experiments, and/or synthetic augmentation. Models may be trained on temporal sequences or aggregated feature representations, and may incorporate transfer learning or fine-tuning from pre-trained models.

In one embodiment of the invention, model outputs in step (650) may include probabilistic estimates, categorical classifications, anomaly scores, or continuous severity indices corresponding to respiratory events (e.g., apnea, hypopnea, distress states), and may support both real-time inference and predictive forecasting.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for monitoring, detecting, and mitigating respiratory irregularities in a patent, comprising:
a contained enclosure;
a plurality of sensors configured to monitor physiological and behavioral characteristics of the patient, wherein the plurality of sensors comprises at least a blood oxygen detector;
an oxygen-air delivery module configured to automatically deliver an oxygen-air mixture to the patient, wherein the oxygen-air delivery module supplies an oxygen-air mixture to the contained enclosure, wherein the oxygen-air delivery module further comprises:
an oxygen-air mixture recycling unit configured to recirculate air from within the contained enclosure;
a conduit configured to connect the oxygen-air mixture recycling unit to the contained enclosure;
a sterilization unit located within the conduit and configured to sterilize the oxygen-air mixture;
a fan configured to maintain and adjust the air flow into the enclosure;
a physical stimulation module configured to stimulate the patient; and
a processor configured to communicate with the plurality of sensors, and to control the oxygen-air delivery module and the physical stimulation module.

2. The system of claim 1, wherein the plurality of sensors comprises at least one of the following sensors: an optical sensor configured to capture optical data associated with the patient, a barometric pressure sensor, a temperature sensor, an audio sensor, an accelerometer, or a gyroscope.

3. The system of claim 1, further comprising a caregiver notification module configured to generate an alert upon a status change of the oxygen-air delivery module or the physical stimulation module.

4. The system of claim 1, wherein the oxygen-air delivery module further comprises a nebulizer.

5. The system of claim 1, wherein the oxygen-air delivery module further comprises an oxygen actuator configured to adjust a concentration of oxygen in the oxygen-air mixture.

6. The system of claim 1, wherein the physical stimulation module comprises at least one of a vibration device, an airflow emitter, an auditory stimulus device, or a visual stimulus device.

7. The system of claim 1, further comprising a data storage module configured to record sensor data and system operation actions.

8. The system of claim 1, wherein the sterilization unit comprises an ultraviolet light source.

9. The system of claim 8, wherein the sterilization unit further comprises at least one air filter positioned downstream or upstream of the ultraviolet light source.

10. The system of claim 9, wherein the air filter comprises high-efficiency particulate air (HEPA) filter.

11. The system of claim 1, wherein the processor is configured to control the oxygen-air delivery module based on a result from a machine learning model, wherein the machine learning model comprises deep neural networks, kernel-based methods, and ensemble techniques.

12. The system of claim 3, wherein the caregiver notification module is configured to transmit the alert to at least one of a display, a mobile device, or a remote monitoring station.

13. A method for detecting, monitoring, and mitigating respiratory irregularities in a patient, the method comprising:
continuously monitoring physiological and behavioral characteristics of the patient using a plurality of sensors;
analyzing, by a processor, measurements from the plurality of sensors;
determining, based on the analyzing, whether an impending or early-stage respiratory irregularity is present; and
automatically initiating a first mitigation response by delivering an increased oxygen-air mixture to the patient via an oxygen-air delivery module when the impending or early-stage respiratory irregularity is detected.

14. The method of claim 13, wherein the physiological and behavioral characteristics comprise at least one of blood oxygen level, respiratory activity, chest movement, abdominal movement, body orientation, body temperature, acoustic signals, eye movement, or patient restlessness.

15. The method of claim 14, wherein determining an impending or early-stage respiratory irregularity comprises:
determining whether a blood oxygen level drops below a predefined threshold;
determining whether one or more additional physiological or behavioral characteristics of the patient are abnormal when the blood oxygen level drops below the predefined threshold; and
determining, based on the analyzing, that the impending or early-stage respiratory irregularity is present.

16. The method of claim 14 wherein determining whether an impending or early-stage respiratory irregularity is present comprises evaluating an output of a machine learning model, wherein the physiological and behavioral characteristics are provided as inputs to the machine learning model.

17. The method of claim 13, further comprising initiating a second mitigation response when the first mitigation response fails to resolve the respiratory irregularity within a predetermined time period, wherein the second mitigation response comprises applying at least one of vibration, airflow, light, or sound stimuli.

18. The method of claim 13, further comprising sterilizing the oxygen-air mixture prior to delivering the oxygen-air mixture to the patient.

19. The method of claim 18, wherein sterilizing the oxygen-air mixture comprises exposing the oxygen-air mixture to ultraviolet radiation.

20. The method of claim 18, wherein sterilizing the oxygen-air mixture comprises filtering the oxygen-air mixture by a high-efficiency particulate air (HEPA) filter.
